(19) United States
(12) Patent Application Publication
(10) Pub. No.: US 2005/0149100 A1
(43) Pub. Date: Jul. 7, 2005

(54) Cervical canal dilator

(75) Inventors: Jonathan Foltz, Wilton, CT (US); David Reed, New Canaan, CT (US); David Olive, Middleton, WI (US)

Correspondence Address: Stephen E. Feldman, P.C., Suite 701, 12 East 41st Street, New York, NY, 10017, US

(73) Assignee: O.S. Technology LLC

(21) Appl. No.: 11/050,144

(22) Filed: Feb. 2, 2005

Related U.S. Application Data
(63) Continuation of application No. 10/317,351, filed on Dec. 12, 2002.

(51) Int. Cl. A61M 29/00

(52) U.S. Cl. 606/192

(57) ABSTRACT

A cervical canal dilating assembly and method of use are shown. The dilator assembly includes a plastic shaft, a first inflatable member, and a second inflatable member. The shaft can range from being rigid to being highly flexible. The second inflatable member is fabricated of a non-elastic material and is configured to have a maximum inflatable diameter. The second inflatable member is configured to have a predetermined maximum inflatable diameter ranging from 4 to 20 mm. The dilating assembly can also be at least partially covered by a sheath. A control system includes means for measuring pressure configured for at least monitoring the pressure of the second inflatable member. A wire can be used in selected configurations to stiffen and shape the shaft. In operation, the initial penetration of the dilating assembly into the uterus uses a wire for increased stiffness. The dilating assembly is then forwarded through the remainder of the cervical canal. The first inflatable member is expanded in the uterus after being uncovered by the sheath. The second inflatable member is positioned in the cervical canal and gradually inflated to a predetermined maximum diameter.

### BACKGROUND

1. Technical Field

The present disclosure relates to devices for cervical dilation. More particularly, the present disclosure relates to devices for cervical dilation using inflatable members.

2. Background of Related Art

Cervical canal dilators having tubular shafts with inflatable expanding members, such as balloons, are well known and have functions ranging from incontinence catheters to assisting in childbirth. One or more balloons are inflated after positioning the dilator through the cervical canal. The expanded balloons secure the tubular shaft in position and, in combination with the function of dilation, frequently provide the critical passageway for fluid passage, diagnostic devices, or treatment instruments.

A device and process for cervical dilation prior to an abortion is described in U.S. Pat. No. 3,848,602 to Gutnick. The device includes an elongate shaft member formed of firm flexible material and terminating in a distal end with a rounded tip. A proximal end has three discrete conduits connected with three discrete channels. One of the channels is connected with the distally positioned inflatable anchor member and another is connected with a proximally positioned inflatable dilating member. The distal end portion defines a plurality of apertures and is connected with a third channel for fluid flow from the ambient through the dilator. The fluid exits the proximal end portion.

Gutnick teaches an inflatable dilating member having an expandable peripheral membrane, like a balloon, that may be reinforced with a scrim of Dacron to ensure the cylindrical form is maintained throughout the dilation. Gutnick further describes having at least three different sizes of inflatable dilating members with each inflatable dilating member being configured for maximum flexibility of application. The inflatable dilating members are preferably configured to be expandable substantially in excess of the minimum degree of cervical dilation necessary for the abortion.

Gutnick, however, is limited by the combination of the structural material of the dilating member being elastic and the inflation process of supplying a controlled volume of liquid to the elastic dilating member to produce an inflated diameter of the elastic member which is stated in one example as “about somewhat greater than 10 mm and preferably expand up to about 15 mm.” Thus, the structure of the Gutnick elastic dilating member and the inflation process thereof is limited in its ability to accurately produce a specific or controlled desirable maximum inflation diameter. The Gutnick method to determine the diameter of inflation relative to a given volume is not directly measured and thus is highly subjective, vulnerable to varying lengths of conduits and fluid losses and is therefore also vulnerable to being overly expanded and damaging the cervix.

In U.S. Pat. No. 4,664,114 to Ghodsian, a dilator for a cervical canal is described having a double walled cylindrical shaft member open at both ends. The two walls of the tubular cylindrical shaft members are coaxial and separated by a first hollow conduit and a second hollow conduit positioned as spacers between the shaft members. The shaft member has a frontal end including a first inflatable membrane. A second inflatable member is positioned on the exterior wall of the cylindrical shaft. The hollow conduits are separately coupled to the inflatable membranes.

A solid cylindrical member or stylet is positionable within the inner lumen. The stylet has a front end having a blunt tip configured to extend beyond the frontal end of the shaft member. A cap member positioned on an opposing end or proximal end of the stylet provides an airtight seal about the double walled shaft member. In operation, the stylet can be removed from the inner lumen and the inner lumen employed as a passageway for various medical implements.

A disc member is positioned on the shaft member and employed to limit the penetration of the dilator into the cervix. An exterior dampening means or spring abutting the disc member is also at least partially positioned inside the vagina of a patient.

The Ghodsian dilator is a complex arrangement requiring the blunt tip of the stylet to extend beyond the front end of the dilator and the cap to provide a sealing interface during penetration. In addition, the position of the disc member relative to the second inflatable member can cause the positioning of the dilator within the uterus to vary depending upon the length of the cervix. This can result in a partial or an uneven dilation of the cervix because the combination of the length of the second inflatable member relative to its placement in the cervix can be too short to adequately treat all cervixes. Finally, the disc member limits the visualization of the positioning of the dilator into the cervical canal adding further risk of harm to the patient.

In U.S. Pat. No. 5,104,377 to Levine, a device and method for accessing the uterus for manipulation or treatment is described. The device includes a shaft having a distally positioned first expandable distal member and a proximally positioned second inflatable member. The first expandable member is positioned in the uterus and the second inflatable member is inserted partially through the cervical canal such that the first expandable member and the second inflatable member are positioned to exert a clamping force towards each other to secure the shaft in the cervix. Only the portion of the second inflatable member that is outside of the uterus expands.

The shaft distal end is inclined at an angle relative to the remainder of the shaft from between 15 to 25 degrees. The shaft defines three longitudinally aligned lumens. The first and second lumens are in communication with a first expandable member and a second inflatable member, respectively. A third or central lumen runs the full length of the shaft ending in a distal orifice. The shaft is a tube made of extruded vinyl or polycarbonate. The shaft is desired to be relatively rigid in order to provide support for uterine manipulation, but can be adjustably stiffened by installing a stainless steel rod within the central lumen. The rod is envisioned as having any length within the lumen, but it is desirable that the rod extend beyond the proximal portion such that the rod lies at least partially within the cervix. The rod is secured in position at the proximal end of the device by a luer fitting.

Levine is limited by its inability to dilate the cervical opening beyond the diameter of the shaft. In addition, the limited range of the angle of inclination of the distal end between 15 and 25 degrees also inhibits the flexibility in which Levine can be applied due to natural variations in the orientation of the cervix to the axis defined by the vagina. In addition, the balloons or first inflatable member and second expandable member lack the ability to provide an indication as to how much compressive pressure they are applying against the cervix while securing the shaft. Further, the metal rod is stated as being selectively employed to stiffen the shaft for uterine manipulation. The ability of the metal rod to penetrate beyond the tip of the shaft and damage the uterus also presents a potential safety hazard.

In U.S. Pat. No. 5,947,991 to Cowan a cervical ripening device for inducing labor is described. The device includes a single balloon positioned on a shaft. The balloon in the expanded position has ends opposing ends having diameters greater than a diameter of the central section diameter. The balloon is thus a modified cylinder having concave sides forming an hourglass type shape. The shaft is open at both ends.

The ability of the ripening device of Cowan to provide uniform pressure along the length of the cervix in all situations is questionable. The application of this shape of device may unevenly dilate the cervix by over dilating the edges and under dilating the central portion. Under dilating can complicate the passage of instruments. Uneven dilation can cause discomfort to the patient and damage to the cervix. Further, the shape of the balloon inhibits the ability of the physician to monitor the amount of dilation being achieved by the device. Overly dilating the cervix can cause damage to the cervix.

A continuing need exists for a cervical canal dilator including a dilating member having a predetermined maximum diameter of inflation and a shaft having a range of stiffness suitable for differing patient internal geometries and including a flexible shaft capable of being shaped and having a variable stiffness suitable for accommodating differing patient internal geometries.

### SUMMARY

A cervical canal dilator is described including an elongate tubular shaft having an outer surface, a distal end portion, and a proximal end portion. The distal end portion and the proximal end portion define a first longitudinal axis. The shaft defines at least two internal lumens including a first internal lumen and a second internal lumen aligned with the longitudinal axis. The distal end portion defines a tapered tip.

A first inflatable member is positioned on the outer surface of the distal end portion of the shaft. The first member is in fluid communication with the first lumen and is configured for being positioned between a deflated position and an inflated position.

A second inflatable member is positioned on the outer surface of the distal end portion of the shaft and proximal to the first member. The second member is in fluid communication with the second lumen and is configured for being positioned between a deflated position and a predetermined maximum diameter of inflation. The second member is fabricated of a non-elastic material configured to limit the inflation of the second member to the predetermined diameter of maximum inflation.

A control system is connected with the at least two lumens and includes means for a fluid system. The means for the fluid system is in fluid communication with at least the two lumens.

### BRIEF DESCRIPTION OF THE DRAWINGS

Preferred embodiments of the presently disclosed cervical canal dilator are described herein with reference to the drawings, wherein:

FIG. 1A is a side view of a distal end portion of one preferred embodiment of a cervical canal dilator in a first position constructed in accordance with the present disclosure;

FIG. 1B is a side view of the distal end portion of the cervical canal dilator of FIG. 1A with a sheath in place over the inflatable members;

FIG. 2 is a cross-sectional view along lines 2-2 of the cervical canal dilator of FIG. 1A;

FIG. 3 is a side view of the cervical canal dilator of FIG. 1A in a second position constructed in accordance with the present disclosure;

FIG. 4A is a cross-sectional view along lines 4A-4A of the distal end portion of a second embodiment of the cervical canal dilator of FIG. 1A constructed in accordance with the present disclosure;

FIG. 4B is a cross-sectional view along lines 4B-4B of the distal end portion of the second embodiment of the cervical canal dilator of FIG. 4A constructed in accordance with the present disclosure;

FIG. 5 is a side view of a cervical canal of a patient and the cervical canal dilator of FIG. 1A in the first position;

FIG. 6 is a side view of the cervical canal and the cervical canal dilator of FIG. 1A with a first inflatable member in the second position;

FIG. 7 is a side view of the cervical canal and the cervical canal dilator of FIG. 1A with the first inflatable member and a second inflatable member in the second position; and

FIG. 8 is a side view of a distal end portion of a third embodiment of the cervical canal dilator of FIG. 1A constructed in accordance with the present disclosure.

### DETAILED DESCRIPTION OF THE EMBODIMENTS

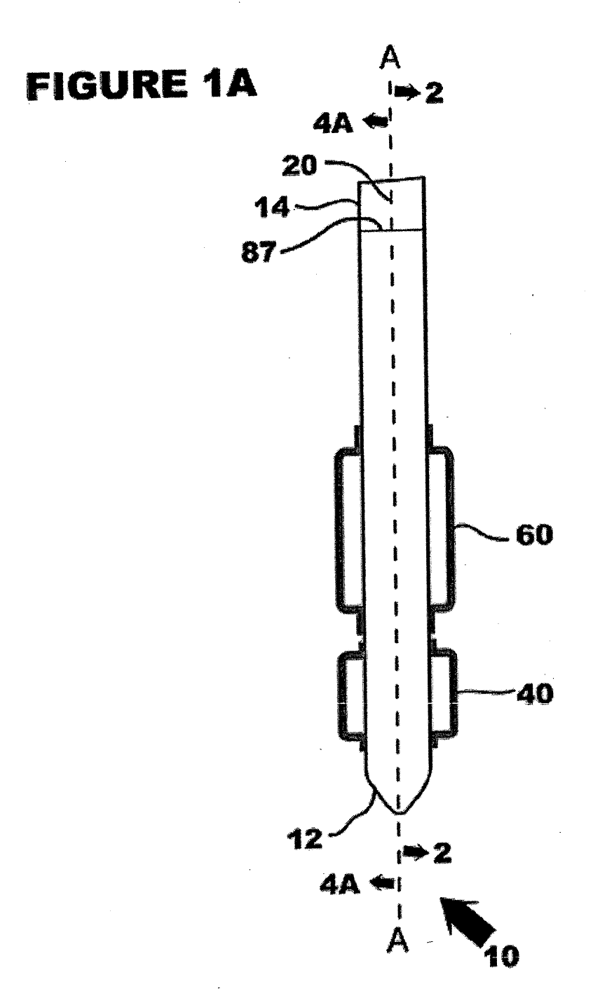

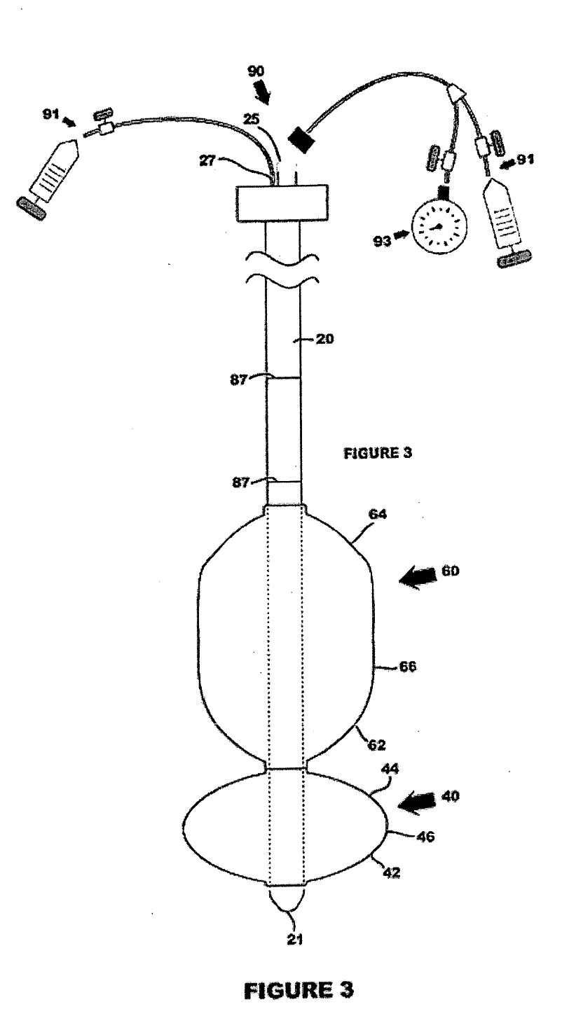

Referring now in specific detail to the drawings in which like referenced numerals identify similar or identical elements throughout the several views, and initially to FIG. 1A, a novel cervical canal dilator assembly 10 is shown having a shaft 20, a first inflatable member 40, a second inflatable member 60 and a control system 90 (see FIG. 3). Cervical canal dilator assembly 10, hereinafter referred to as “dilator 10” has a distal end 12 and a proximal end 14 defining a longitudinal axis-A.

In FIG. 1B, the novel cervical canal dilator assembly 10 is shown having a sheath 80. Sheath 80 includes markings 88 for correlating the position of the first inflatable member relative to the proximal end of the sheath. Shaft 20 includes markings 87 for correlating the position of sheath 80 relative to the inflatable members. Dilator 10 is adapted for use by a physician and is configured as a readily useable disposable device having a reduced cross-sectional dimension of less than 4 mm. Additional features of dilator 10 are intended to reduce the risk of trauma during the insertion and dilation of a cervical canal of a patient by the physician.

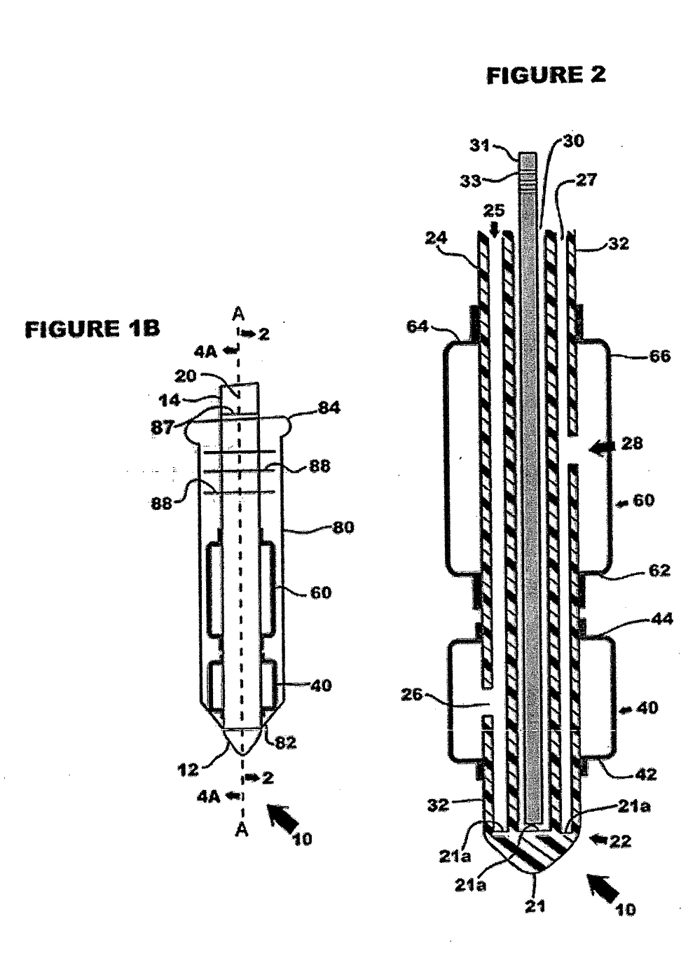

Referring now to FIGS. 1A, 1B, 2, and 3, shaft 20 has a distal end portion 22 and a proximal end portion 24 aligned with first longitudinal axis-A. Distal end portion 22 includes a tip 21 having a solid circular base 21a and a tapered or conical outer shape. Tip 21 is advantageously shaped for ease of insertion into the cervical canal of the patient. Shaft 20 has a cylindrical outer surface 32. Proximal end portion 24 is connected with control system 90.

Shaft 20 is fabricated of a medical grade plastic or composite material. Shaft 20 can have a flexible, semi-rigid, or rigid configuration. Flexible shaft 20, in one preferred embodiment, is highly flexible to the point of becoming at least partially floppy and having only a moderate degree of stiffness along longitudinal axis-A. The rigid construction can be straight or include an arcuate portion encompassing at least part of distal end portion 22. The semi-rigid configuration is flexible and/or bendable with a memory such that semi-rigid shaft 20 can retain a specifically defined shape. The term flexible shaft 20, as noted herein, refers to shafts 20 having flexible configurations selectively augmented by a wire for shaping and/or stiffening.

Shaft 20 is a solid shaft 20 defining inner walls for a first lumen 25, a second lumen 27, and a third lumen 30. First lumen 25 is connected with a port 26 positioned through outer surface 32 for fluid communication with first inflatable member 40. Similarly, second lumen 27 is connected with a port 28 positioned through outer surface 32 for communication with second inflatable, member 60. Lumens 25, 27, and 30 are terminated and sealed on their distal ends by base 21a of tip 21 and connected with control system 90 on their opposing proximal ends.

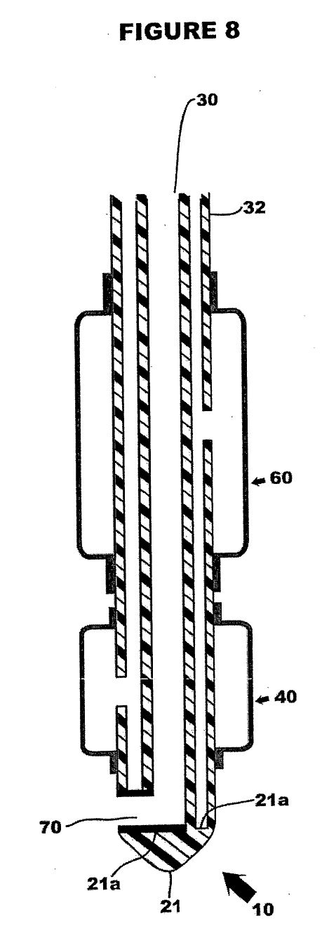

Third lumen 30 is preferably positioned between lumens 25 and 27 and axially aligned with the longitudinal axis-A. Third lumen 30 can be configured to only define a proximal end port connected with control system 90 or to define a side port 70 in outer surface 32 (see FIG. 8) distal to first member 40 and proximal to tip 21. Port 70 allows fluid communication with the inside of the uterus in applications, such as for example, prior to completion of cervical dilation and removal of the dilator. Uses of this channel can also include diagnostic readings or infusion of therapeutic agents to treat pain and bleeding within the uterus.

Flexible shaft 20 includes a wire 31, an elongate element, suitably sized for positioning in one of the lumens of shaft 20. When wire 31 is positioned in one of the lumens of shaft 20, a distal end of wire 31 abuts base 21a and a proximal end extends from shaft 20. Wire 31 is configured for ease of removal and replacement in one of the lumens. Wire 31 provides an improved degree of stiffness along longitudinal axis-A of flexible shaft 20. In addition, wire 31 assists in the shaping of the highly flexible configuration of shaft 20.

In one preferred embodiment, wire 31 is positioned in lumen 30. In another preferred embodiment, wire 31 is positioned in the first lumen 25 or second lumen 27. When wire 31 is positioned in lumen 25 or 27, wire 31 is preferably removed prior to the application of fluid to the lumens, but wire 31 can be selectively retracted at any time prior to the application of fluid to lumen 25, 27, or 30.

Wire 31 is made of a bendable material with a memory such that shaft 20 can be shaped for insertion in a cervix oriented at an angle to the patient's vagina. Shaping wire 31 and/or shaft 20 includes bending at least the distal end portion 22 of shaft 20 to replicate the approximate angle between the cervical opening and the vaginal canal such that the distal end portion is generally perpendicular to the cervical opening. Wire 31 could also be preformed to have an arcuate shape or arcuate bend based on a shaped memory material. Arcuate bent wire 31 is retractable from shaft 20 so that the flexibility of shaft 20 can be selectively controlled when shaft 20 is a flexible member.

The stiffness of flexible shaft 20 and in particular, distal end portion 22, can be controlled by partially with drawing or retracting wire 31 from contact with base 21 a and/or distal end portion 22. In this manner, when distal end portion 22 is positioned inside the cervical canal, for example, distal end portion 22 can be made less stiff than the portion of shaft 20 inside the vagina of the patient. Wire 31 can be selectively retracted so the portion of shaft 20 distal to member 40 or distal to member 60, for example, is more flexible than the remaining proximal portions of shaft 20. Wire 31 can be adjustably bent along its full length to bend shaft 20 in an at least partially arcuate shape that includes, for example, a purely arcuate shape or a combination of angled and arcuate shapes prior to or after positioning wire 31 in shaft 20. Wire 31 is retracted to predetermined positions within shaft 20 using markings 33 positioned on the proximal end of wire 31. Wire 31 is preferably made of a medical grade metal and retains its axial stiffness while bent.

First inflatable member or member 40 is positioned proximal to and in juxtaposition with tip 21 and has a compressed or folded annular shape in a first position. Member 40 has a proximal end 44 and a distal end 42 and is fabricated of a stretchable or non-stretchable medical grade rubber, plastic, or composite material suitable for uterine applications. In one preferred embodiment, when a fluid is supplied by control system 90 through lumen 25 to member 40, member 40 expands to a shape having an outer surface 46 of an oblate spheroid with a first shorter axis aligned with longitudinal axis-A and a second wider diameter or axis generally perpendicular to longitudinal axis-A. The second diameter of member 40 can be larger than the diameter of second inflatable member 60.

Member 40 has a range of sizes having different inflated second diameters. Thus, member 40 provides a mechanism for ensuring that unintended proximal travel through the cervical canal by dilator 10 from its uterine position is precluded and the cervical canal dilator remains in position during the dilating process.

In another embodiment of the invention, inflatable member 40 has the same or smaller second diameter as inflatable member 60, so that shaft 20 can discharge automatically from the cervix when the desired dilation is achieved.

The length of member 40 will be in the range of 1 to 2 cm to minimize the portion of the dilator assembly positioned within the uterus. This will prevent the device from damaging the edges of the uterus when the uterus is oriented at an angle to the cervix.

First inflatable member 40 can be inflated to a desired diameter by means for a fluid system 91 supplying a controlled amount of fluid, the use of means for measuring pressure, or combinations thereof.

Second inflatable member or member 60 is positioned proximal to and in juxtaposition with member 40 and has a compressed or folded annular shape in the first position. The first position for members 40 and 60 is a compact position intended to minimize the dimension in the radial direction from the longitudinal axis-A. Member 60 has a distal end 62 and a proximal end 64. Member 60 is fabricated of a non-stretchable or non-elastic type medical grade plastic or composite material suitable for internal applications. Member 60 can have a membrane or a woven configuration. When a fluid is supplied by control system 90 through lumen 27 and the port to member 60, member 60 inflates both radially and axially into an annular shape having a cylindrical outer surface 66 generally parallel to the longitudinal axis-A. Distal end portion 62 and proximal end portion 64 have generally tapered spheroid shapes. When in the inflated position the first member 40 and second member 60 are at least partially in direct contact in order to ensure uniform dilation including the distal end of the cervix, which is the most difficult portion of the cervix to dilate.

Member 60 comes in a range of predetermined maximum diameters of inflation such as, but not limited to 4 mm to 20 mm. The length of member 60 is suitable for extending at least the length of a cervical canal of the patient. Member 60 is configured for uniformly inflating along its length such that the cervix is uniformly dilated as member 60 is inflated to its predetermined maximum diameter of inflation. The predetermined maximum diameter of inflation or maximum inflatable diameter is defined herein as the diameter defined by the configuration of non-elastic second member when fully inflated.

Second member 60 is fabricated and/or constructed of non-elastic material having sufficient strength such that upon reaching its predetermined maximum diameter at full inflation, additional fluid pressure communicated to member 60 will increase the pressure within member 60, but the diameter of member 60 remains fixed. The fixed maximum diameter along the axis perpendicular to longitudinal axis-A of member 60 also functions to reduce the risk of over expanding the cervical canal due to over inflation of the dilating member. Member 60 has a suitable length to ensure it will encompass the full length of a patient's cervical canal.

Member 60 will preferably be in the range of approximately 4 cm to 5 cm to dilate the full length in the majority of female cervixes while minimizing the portion of member 60 inflated within the vagina where it could interfere with visualization of the outer edge of the cervix and to monitor the progress of dilation.

Sheath 80 is a thin layer of medical grade low outer surface friction plastic material having a first position at least partially covering dilator assembly 10 including member 40, member 60, and at least the distal end portion 22 of shaft 20. Sheath 80 can be a shrink wrapped layer or a loosely conforming layer, for example. Sheath 80 has a distal end 82 and a proximal end 84. Distal end 82 is positioned over tip 21 and can include perforations, serrations, or indentations to facilitate sheath 80 in stretching or splitting so that it can be removed from shaft 20, member 40, and member 60.

Distal end 82 is configured to stretch or at least partially separate into segments upon the retraction of sheath 80 proximally such that tip 21 extends through distal end 82 and first member 40 and second member 60 are selectively retracted or uncovered from sheath 80. Sheath 80 is adapted to be retracted proximally along longitudinal axis-A. Sheath 80 can be coated with a lubricating material suitable for uterine applications such as a hydrophilic material to allow for an easy and rapid insertion into the cervical opening.

Sheath 80 has markings 88 positioned at intervals along proximal end 84 to indicate the depth of penetration of tip 21, member 40, and member 60 into and/or through the cervical canal. Additional markings 87 on shaft 20, for example, indicate how far sheath 80 has been retracted and to thereby ensure that sheath 80 is clear of member 40, for example, prior to inflation.

Control system 90 includes means for a fluid system 91. Means for a fluid system 91 can include items typically found in pressurized fluid systems such as, but not limited to a closed circuit of lines, connectors, valves, supply and exhaust reservoirs, pumps, pressure gauges, and safety devices such as pressure release valves. Means for a pressure system 91 includes separate pressure systems for first member 40 and second member 60. Items such as the reservoir and pump, for example can be a single item such as a syringe having suitable fluid capacity or separate items.

Means for a fluid system 91 includes means for measuring pressure 93, such as a pressure gauge, in fluid communication with second inflatable member 60. Means for measuring pressure 93 is configured to measure the pressure within second member 60, and by relation, pressure within the cervical canal. The ability to measure the pressure within member 60 allows the physician to have an improved method of control over the dilation process and determining when the maximum dilation of second member 60 is achieved. Means for measuring pressure 93 and means for a fluid system 91 are configured for precisely controlling the amount of pressure applied to member 60 and thereby to the patient during the dilation process.

Means for measuring pressure 93 can include an adjustable tolerance or range setting such that if the pressure drops below or rises above a particular range then an alarm or warning is provided. It is further envisioned that the pressure in member 60 can be made to increase or decrease at a predetermined rate by increments over time.

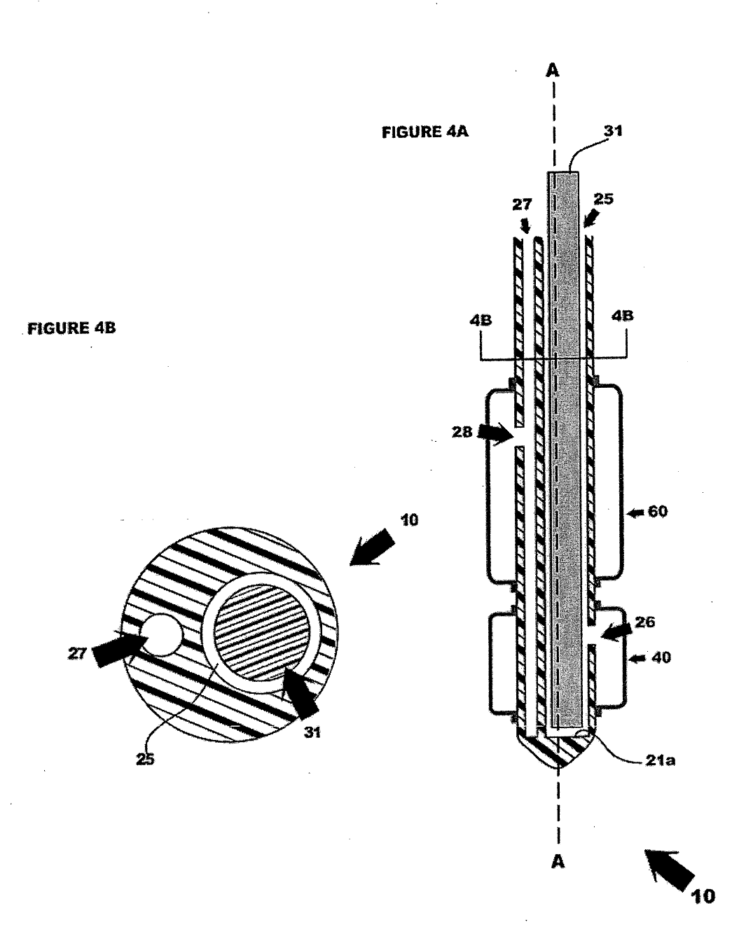

Referring now to FIGS. 3, 4A, and 4B, in another preferred embodiment, cervical canal dilator 10 has a shaft 20 including only first internal lumen 25 and second internal lumen 27. First lumen 25 has a diameter suitable for being in fluid communication with first member 40 through port 26 and for the positioning of wire 31 when shaft 20 is in the flexible configuration. Second lumen 27 is in fluid communication with the second member 60 through port 28. In this embodiment, the distal end of wire 31 is initially positioned abutting base 21 a and the proximal end extends from shaft 20. Wire 31 functions to provide an improved element of stiffness axially, along the longitudinal axis-A. Wire 31 is readily positioned and removed from lumen 30, but is removed prior to the connecting first lumen 25 with control system 90 for fluid communication. In the rigid or semi-rigid configurations not requiring wire 31 to augment stiffening of shaft 20, lumens 25 and 27 are not necessarily sized for the positioning of wire 31.

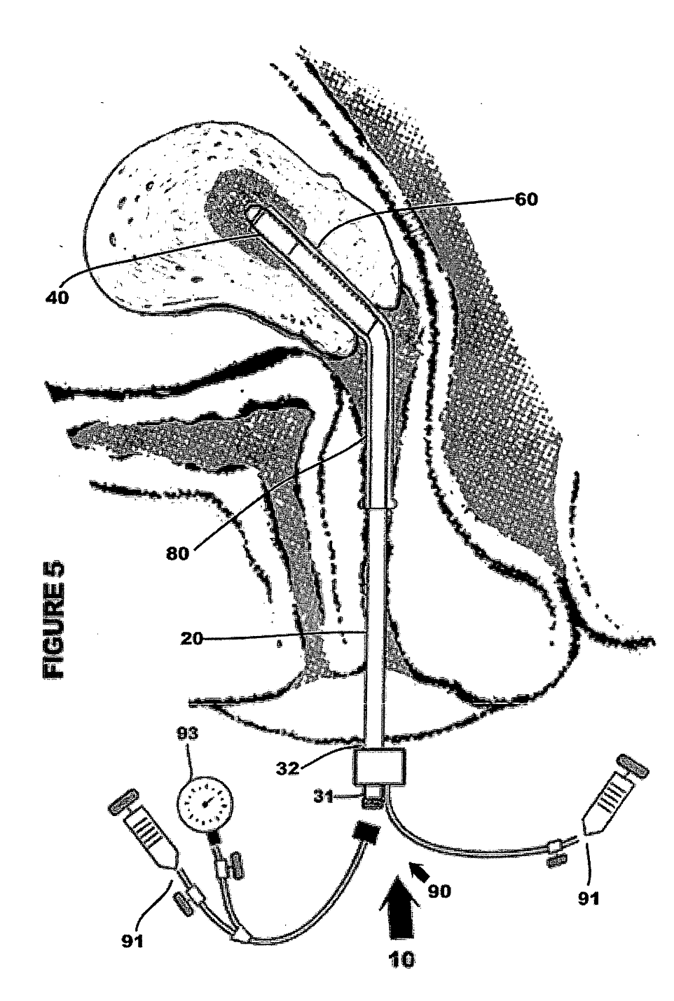

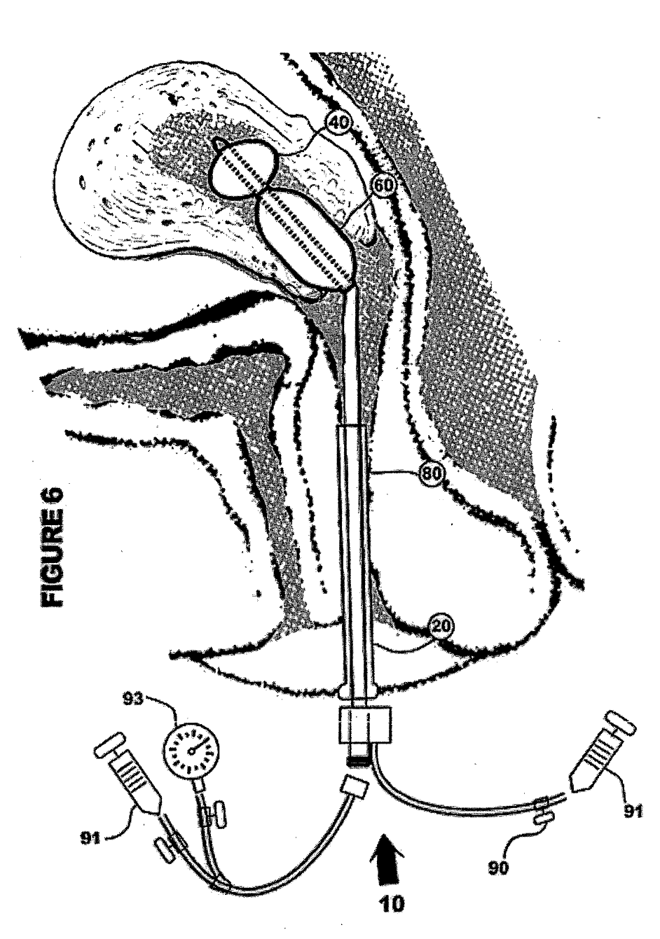

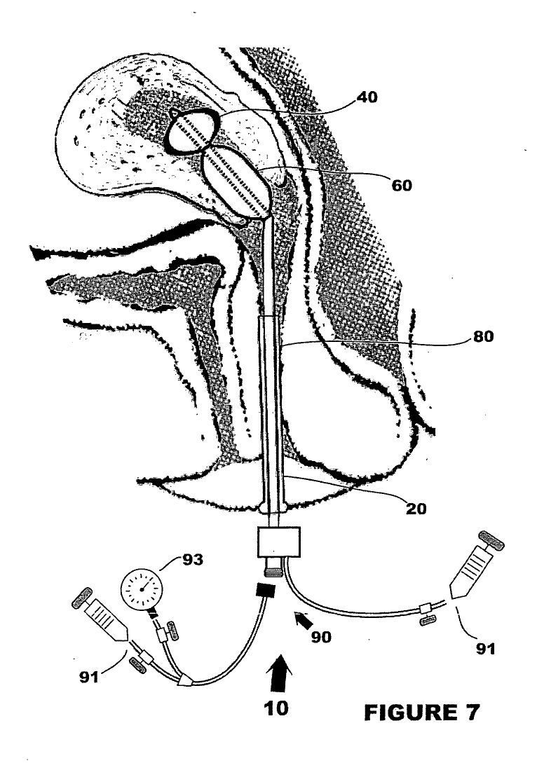

As shown in FIGS. 1B, 2, 4A, and 5-7, in operation, the physician selects a desired configuration of cervical canal dilator 10 for application with the patient for the dilation of the patient's cervical canal to the predetermined maximum diameter. This process includes evaluating the patient internal geometries, such as the angle between the vagina and the cervical opening, to determine whether the rigid, semi-rigid, or flexible shaft 20 configuration will be utilized. Cervical canal dilator assembly 10 is initially in the first position with first member 40 and second member 60 compactly positioned against outer surface 32. Depending upon the configuration, cervical canal dilator 10 is covered by sheath 80 compactly positioned against outer surface 32, first member 40, and second member 60.

When utilized, wire 31 positioned in one of the lumens such that the distal end of the wire abuts base 21a and the proximal end of the wire extends from shaft 20 to provide accessibility to the physician. When wire 31 is made of bendable material, it can be shaped to the desired angle or arcuate orientation before or after positioning in flexible shaft 20. The bending of wire 31 is preferably performed when dilator 10 is in the first position with wire 31 positioned fully in shaft 20 such that the distal end of wire 31 is abutting base 21a. Wire 31, for example, can be shaped for insertion in a cervix that is oriented at an angle to the patient's vagina.

Dilator 10 in this position has a diameter less than 4 mm and is considered suitable for application in all cervixes. Dilator 10 is positioned at least partially into the cervical opening. Once distal end 12 has been inserted a predetermined distance, such as approximately 4 mm, into the cervix, wire 31 when present can be selectively retracted from shaft 20 such that when tip 21 is positioned inside the cervical canal, distal end 22 can be made advantageously less stiff than the portion of shaft 20 inside the vagina. The reduction in stiffness can reduce the risk of accidental damage to the cervix.

Distal end 12 is then inserted further into the cervix a second predetermined distance, such as for example three centimeters, to position first inflatable member 40 within the uterus. With wire 31 removed, shaft 20 retains sufficient axial rigidity for forwarding through the cervical canal and yet is suitably flexible or floppy to drastically reduce the likelihood of inadvertently perforating the uterine wall. The penetration through the cervical canal can be aided by a hydrophilic material, positioned on tip 21 or the surface of sheath 80. When configured with sheath 80, markings 88 positioned at intervals along proximal end 84 indicate the depth of penetration of tip 21, member 40, and member 60 into and/or through the cervical canal.

When present, sheath 80 is then retracted proximally along longitudinal axis-A to uncover first inflatable member 40 using the correlation markings on sheath 80 and/or shaft 20. Using control system 90, means for a fluid system 91 sends a predetermined volume of fluid, such as, but not limited to a saline solution, to inflate member 40 and initiate placing dilator 10 from the first position to the second position. A syringe or another pressurizing and reservoir system can be used to inflate member 40. Dilator 10 is then moved proximally until member 40 engages the internal edge of cervix.

Sheath 80, when included in the configuration of dilator assembly 10, is then retracted proximally along longitudinal axis-A to uncover second inflatable member 60 using the correlation markings 87 on sheath 80 and/or shaft 20. Control system 90, including means for a fluid system 91 and means for measuring pressure 93, is used to inflate second member 60 from the first position to the predetermined maximum diameter of inflation or second position. Member 60 expands both axially and radially initially in a uniform manner into an elongate cylindrical shape having spheroid distal and proximal ends. The inflation of member 60 axially brings member 60 at least partially in direct contact with member 40.

The inflation of member 60 continues after the axial limit is reached in a uniform radial inflation until the predetermined maximum diameter of inflation is achieved. This advantageously uniformly inflates the diameter such that a uniform pressure is placed along the cervix and limits the dilation of the cervix to the desired diameter. The inflation of member 60 is typically done in a series of graduated steps and is completed by the positioning of dilator 10 in the second position. A syringe or another pressurizing and reservoir system can be used to inflate member 60. Means for measuring pressure such as a pressure gauge 93 is preferably used to monitor the pressure applied to the second member and cervix during the dilation process and determines when the cervix has expanded in response to the pressure of the second member by a reduction in pressure, for example, as well as determining when second inflatable member has reached its maximum diameter of inflation or second position.

Means for measuring pressure 93 can also be advantageously used to measure the dilation or relaxation of the cervix after an incremental increase of the pressure to second member 60 for dilation. Alternatively, or in combination, member 60 could be inflated to its maximum diameter using a predetermined amount of fluid.

When second member 60 is inflated to its maximum predetermined diameter of inflation and the desired cervical canal dilation is achieved, second member 60 and first member 40 are deflated returning dilator 10 to the approximate diameter of the first position. Dilator 10 is then withdrawn from the patient.

In another preferred embodiment, as shown in FIGS. 3 and 8, cervical canal dilator 10 includes a side port 70 defined in outer wall 32 of third lumen 30. Lumen 30 is in communication with means for fluid system 91. Port 70 is preferably distal to the first inflatable member 40, allowing fluid communication with the inside of the uterus for applications such as but not limited to providing treatment inside the uterus prior to completion of cervical dilation and removal of the catheter. Port 70 also accommodates, for example, the making of diagnostic readings from the inside of the uterus that can be recorded using control system 90. Alternatively, therapeutic agents can be injected through port 70 into the uterus to treat conditions such as cramps or bleeding. Port 70 is in outer surface 32 in order to not interfere with the streamlined low friction shape of tip 21a.

Although the illustrative embodiments of the present disclosure have been described herein with reference to the accompanying drawings, it is to be understood that the disclosure is not limited to those precise embodiments, and that various other changes and modifications may be affected therein by one skilled in the art without departing from the scope or spirit of the disclosure. All such changes and modifications are intended to be included within the scope of the disclosure.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. A method of dilating a cervical canal of a patient, comprising the steps of: providing a dilator assembly having a first inflatable member and a second inflatable member positioned on a shaft, the second inflatable member being fabricated of a non-elastic material and configured to define a uniform maximum diameter for dilating the cervix of a patient, a control system in fluid communication with the first inflatable member and the second inflatable member; positioning the dilator assembly in a first position for penetration into a cervical canal of the patient, making an initial penetration of the cervical canal and positioning the first inflatable member through the cervical canal; placing the dilator assembly in the second position by using the control system for inflating the first member, positioning the inflated first member against the inner side of the cervical canal, using the control system to gradually inflate the second member to a maximum diameter of inflation defined by the second member, the second member being configured to dilate the cervical canal of the patient to the predetermined maximum diameter of inflation of the second member; and deflating the first member and deflating the second member, withdrawing the dilator assembly from the patient.

33. The method of claim 32, wherein the step of providing includes a flexible shaft and the initial forwarding of the dilator assembly into the cervical canal of the patient is performed using a wire.

34. The method of claim 33, wherein the step of positioning further includes bending any portion of the flexible shaft such that the flexible shaft can be shaped to include an at least partially arcuate portion suitable for insertion into a vagina and the cervix of the patient when the cervix is aligned to the vagina at an angle.

35. The method of claim 33, wherein the step of positioning further includes selectively retracting the wire of the shaft to decrease the stiffness of the distal end portion once the tip has been positioned within the cervix.

36. The method of claim 32, wherein the step of placing includes reading a pressure level on a pressure gauge, the pressure level determining the maximum diameter of inflation of the second member has been achieved.

37. The method of claim 32, wherein the step of placing includes reading a means for measuring pressure to determine the cervix has dilated to the predetermined maximum diameter and the dilator assembly can be removed.

38. The method of claim 32, wherein the step of placing includes inflating the first member such that the diameter of the first member is smaller than the diameter of the second member causing the catheter to automatically discharge when the cervix is dilated.

39. The method of claim 32, wherein the step of providing includes the shaft, the first inflatable member, and the second inflatable member being at least partially covered by a sheath.

40. The method of claim 39, wherein the step of placing further includes retracting the sheath from the first member prior to inflating the first member.

41. The method of claim 39, wherein the step of placing further includes retracting the sheath from the second member prior to inflating the second member.

42. The method of claim 33, wherein the step of positioning further includes removing the wire from the dilator assembly upon making the initial penetration into the cervix.

43. The method of claim 33, wherein the step of providing includes a shaft having three lumens, a third lumen defining a passageway parallel to the longitudinal axis and defining a port on the side of the shaft at a point distal to the first inflatable member, the port being suitable for collecting diagnostic data from the inside of the uterus and infusing the uterus with therapeutic agents.

44. (canceled)

45. (canceled)

46. (canceled)

47. (canceled)

48. (canceled)

49. (canceled)
